(19) United States
(12) Patent Application Publication
(10) Pub. No.: US 2002/0143064 A1
(43) Pub. Date: Oct. 3, 2002

(54) Novel use of compounds of the chalcones class

(76) Inventors: Marielle Chiron, Paris (FR); Cecile Combeau, Chaville (FR); Patrick Mailliet, Fontenay Sous Bois (FR)

Correspondence Address: Finnegan, Henderson, Farabow,, Garrett & Dunner, L.L.P., 1300 I Street, N.W., Washington, DC, 20005-3315, US

(21) Appl. No.: 10/013,500

(22) Filed: Dec. 13, 2001

(30) Foreign Application Priority Data
Dec. 13, 2000 (FR) 00 16197

(51) Int. Cl. A61K 31/135

(52) U.S. Cl. 514/646

(57) ABSTRACT

Process for inhibiting vascularization of a tumor mass comprising administering, in an amount effective to inhibit vascularization, at least one chalcone of formula (I):

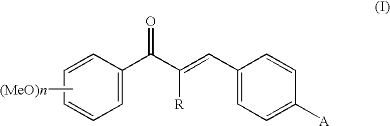

wherein:

R is chosen from a hydrogen atom, a methyl group, an ethyl group, a chlorine atom, and a bromine atom,

A is an NR₁R₂ group, wherein R₁ and R₂, which may be identical or different, are each chosen from a methyl group and an ethyl group, and

n is an integer equal to 2 or 3.

The present invention relates to novel uses of compounds of the chalcones class. It also relates to the use of specific compounds of the class. It furthermore relates to their use in inhibiting the vascularization of a tumor mass and/or to their application in the preparation of a medicament having an antivascular pharmacological property.

The term “inhibition of vascularization of a tumor mass” is understood to mean the destruction of the vessels which irrigate a tumor mass and thus which allow the tumor cells to grow. It is well known scientifically that tumors, in order to grow, require the presence of very extensive vascularization. For some years, researchers specializing in the field of oncology have sought to develop products which would allow these vessels to be destroyed, also known as antivascular products, or which would allow the formation of new vessels to be avoided, also known as antiangiogenic products.

Antiangiogenic products are often cytostatic products, such as, for example, products which block the endothelial growth factors of the vessels, such as VEGFs (vascular endothelial growth factors), the most well-known examples of which are natural polypeptides, such as angiostatin and endostatin. Mention may be made, among antivascular compounds, of non-cytotoxic products, for example, prodrugs for cytotoxic products, such as, for example, those which are disclosed in International Patent Application WO 00/48606, which are phosphates of cytotoxic compounds. These compounds, non-toxic to begin with, have an antivascular effect on the tumor and simultaneously result in the production of specific enzymes which would make possible, with respect to the multivascularization, the conversion of the prodrug into an active drug and, by this twofold effect, the destruction of the tumor.

This effect has been disclosed in International Patent Application WO 00/48606 solely for a class of highly specific compounds, combretastatin phosphates, although the same effect has been mentioned for all the phosphates. In fact, it would appear that phosphatase, an enzyme for the hydrolysis of the prodrug to an active drug, is present and in particular overexpressed with regard to the vascularization of the tumor. No similar effect has appeared to date for the other drugs or prodrugs.

According to patent WO 00/48606, the active drugs, such as, for example, tubulin-binding agents, in their entirety, can reduce the blood flow, but the doses used for this effect are often toxic with regard to normal cells and not particularly toxic with regard to cancerous cells.

It has just been discovered by the inventors that, among the chalcones, some have a tubulin-binding effect and no effect on cell detachment, also known as antivascular effect, and others have an effect on cell detachment and no effect on tubulin. The two effects are therefore not fully correlated and one or more other mechanisms of action are involved and are currently still unknown.

The term “cell detachment” is understood to mean the detachment of the endothelial cells from the vessels, which will result in disorganization of the vessels and consequently stasis of the blood flow and subsequent death of the tumor, essentially as a consequence of the supply of growth factors being severed.

A test for the determination of the detachment of endothelial cells has been developed in order to select products with respect to their “in vitro” activity. Such test was employed in the present invention. By way of this test, the endothelial cells were inoculated in plates, the bottom of which were covered with at least one binding agent, for example, chosen from gelatin, fibronectin, and vitronectin. The inoculated endothelial cells were then cultured. The cultured endothelial cells were treated with a medium comprising the test compound. The cells were then labeled with a fluorescent substance. The detached cells were removed by washing; and the fluorescence of the remaining cells was counted with a fluorimeter.

This test measures the detachment of endothelial cells cultured on substrata based on at least one binding agent, for example, chosen from fibronectin, vitronectin, and gelatin. Generally, one day after the cells were seeded in plates, which comprised, for example, 96 wells, the culture medium was replaced by a medium, which comprised the test compound in the absence of serum. The same preparation was prepared six times at three different concentrations (0.1, 0.3 and 0.6 μM) and the control was prepared six times without addition of antivascular product. After the cells were treated for two hours with the test substance, the cells were labeled with calcein AM (1.6 μg/ml) in culture medium supplemented with 0.1% of BSA. The cells that detached were removed by washing with culture medium comprising 0.1% of bovine serum albumin. 100 μl of medium were then added to each well. The fluorescence of the remaining cells was counted with a fluorimeter. The data obtained is expressed with respect to the control (untreated cells).

The chalcones of formula:

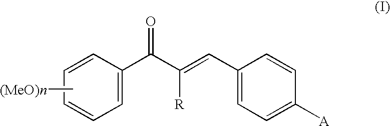

in which:

R is chosen from a hydrogen atom, a methyl group, an ethyl group, a chlorine atom, and a bromine atom,

A is an NR₁R₂ group, wherein R₁ and R₂, which may be identical or different, are each chosen from a methyl group and an ethyl group, and

n is an integer equal to 2 or 3,

showed quite astonishing results with regard to cell detachment and thus in the targeted application, i.e., for antivascular action.

Without implied limitation, the following list of chalcones is representative of the compounds of the present invention:

1-(2,5-dimethoxyphenyl)-3-[4-(dimethylamino)phenyl]-2-methylprop-2-en-1-one,

1-(2,5-dimethoxyphenyl)-3-[4-(dimethylamino)phenyl]prop-2-en-1-one,

1-(2,5-dimethoxyphenyl)-3-[4-(dimethylamino)phenyl]-2-ethylprop-2-en-1-one,

1-(2,5-dimethoxyphenyl)-3-[4-(dimethylamino)phenyl]-2-bromoprop-2-en-1-one,

1-(2,5-dimethoxyphenyl)-3-[4-(dimethylamino)phenyl]-2-chloroprop-2-en-1-one,

1-(2,5-dimethoxyphenyl)-3-[4-(diethylamino)phenyl]-2-methylprop-2-en-1-one,

1-(2,5-dimethoxyphenyl)-3-[4-(diethylamino)phenyl]prop-2-en-1-one,

1-(2,5-dimethoxyphenyl)-3-[4-(diethylamino)phenyl]-2-ethylprop-2-en-1-one,

1-(2,5-dimethoxyphenyl)-3-[4-(dimethylamino)phenyl]-2-bromoprop-2-en-1-one,

1-(2,5-dimethoxyphenyl)-3-[4-(dimethylamino)phenyl]-2-chloroprop-2-en-1-one,

1-(2,3,4-trimethoxyphenyl)-3-[4-(dimethylamino)phenyl]-2-methylprop-2-en-1-one,

1-(2,3,4-trimethoxyphenyl)-3-[4-(dimethylamino)phenyl]prop-2-en-1-one,

1-(2,3,4-trimethoxyphenyl)-3-[4-(dimethylamino)phenyl]-2-ethylprop -2-en-1-one,

1-(2,3,4-trimethoxyphenyl)-3-[4-(dimethylamino)phenyl]-2-bromoprop -2-en-1-one,

1-(2,3,4-trimethoxyphenyl)-3-[4-(dimethylamino)phenyl]-2-chloroprop -2-en-1-one,

1-(2,3,4-trimethoxyphenyl)-3-[4-(diethylamino)phenyl]-2-methylprop -2-en-1-one,

1-(2,3,4-trimethoxyphenyl)-3-[4-(diethylamino)phenyl]prop-2-en-1-one,

1-(2,3,4-trimethoxyphenyl)-3-[4-(diethylamino)phenyl]-2-ethylprop-2-en-1-one,

1-(2,3,4-trimethoxyphenyl)-3-[4-(dimethylamino)phenyl]-2-bromoprop-2-en-1-one,

1-(2,3,4-trimethoxyphenyl)-3-[4-(dimethylamino)phenyl]-2-chloroprop-2-en-1-one,

1-(3,4,5-trimethoxyphenyl)-3-[4-(dimethylamino)phenyl]-2-methylprop-2-en-1-one,

1-(3,4,5-trimethoxyphenyl)-3-[4-(dimethylamino)phenyl]prop-2-en-1-one,

1-(3,4,5-trimethoxyphenyl)-3-[4-(dimethylamino)phenyl]-2-ethylprop-2-en-1-one,

1-(3,4,5-trimethoxyphenyl)-3-[4-(dimethylamino)phenyl]-2-bromoprop-2-en-1-one,

1-(3,4,5-trimethoxyphenyl)-3-[4-(dimethylamino)phenyl]-2-chloroprop-2-en-1-one,

1-(3,4,5-trimethoxyphenyl)-3-[4-(diethylamino)phenyl]-2-methylprop-2-en-1-one,

1-(3,4,5-trimethoxyphenyl)-3-[4-(diethylamino)phenyl]prop-2-en-1-one,

1-(3,4,5-trimethoxyphenyl)-3-[4-(diethylamino)phenyl]-2-ethylprop-2-en-1-one,

1-(3,4,5-trimethoxyphenyl)-3-[4-(dimethylamino)phenyl]-2-bromoprop-2-en-1-one, and

1-(3,4,5-trimethoxyphenyl)-3-[4-(dimethylamino)phenyl]-2-chloroprop-2-en-1-one.

It is known in the prior art that chalcones exert an antitumor activity by interaction with tubulin, as described in the publication by N. Lawrence, A. McGown, S. Ducki and J. Hadfield in Anti-Cancer Drug Design (2000), 15,135-141. At no point in this publication is the antivascular effect of the claimed agents described. Furthermore, only one of the compounds of the publication falls within the formula of the present invention. Additionally, the chalcones described in the publication that are not compounds of the formula of the present invention do not exhibit the antivascular activity highly characteristic of chalcones carrying an alkylamino chain on the phenyl ring gamma with respect to the carbonyl functional group.

The present invention will be more fully described with the help of the following examples, which do not serve under any circumstances to limit the invention.

The chalcones used in the present invention were prepared, for example, by condensation of a trimethoxylated ketone carrying an R group, as defined above, with an aldehyde carrying an A group, as defined above, in a mixture of (C₁-C₄) alcohol and acetic acid in the presence of an organic base, such as piperidine, according to the following scheme:

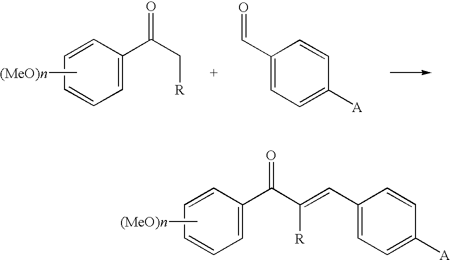

The evaluation of the detachment of endothelial cells in vitro was determined in the following way: HDMEC cells (Human Dermal Microvascular Endothelial Cells, Promocell, c-122102) were cultured in an ECGM-MV medium which comprised 5% of foetal calf serum, growth factors (EGF 10 ng/ml, hydrocortisone 1 μg/ml, 0.4% growth supplement with heparin) and antibiotics (amphotericin 50 ng/ml, gentamicin 50 μg/ml). For the detachment test, the HDMECs were inoculated at 5000 cells in clear-bottomed 96-well plates (Costar) precoated with fibronectin (10 μg/ml) or vitronectin (1 μg/ml) or gelatin. Twenty four hours later, the culture medium was replaced with ECGM-MV 0.1% BSA medium comprising the products shown. The concentrations tested were 0.1, 0.3 and 1 μM for each product. After treating for two hours, the cells were labeled for one hour with calcein (1.6 μg/ml, Molecular Probes) in ECGM-MV 0.1% BSA medium. The detached cells were subsequently removed by washing with ECGM-MV 0.1% BSA medium; 100 μl of medium was added to each well. The fluorescence of the cells which remained attached to the substratum of the well were counted using a fluorimeter, Spectrafluor Plus (Tecan, excitation 485 nm and emission 535 nm). The below data show the mean of six different samples and are expressed as a percentage of the control (untreated cells).

A cell detachment effect of greater than or equal to 15% is regarded as significant.

Table of results

| | | Percentage |
|---|---|---|
| | | of |
| | | detachment |
| | | of HDMEC |
| | | cells |
| | | induced by |
| | | 1 μM of |
| Example | Compound | compound |
| 1 | 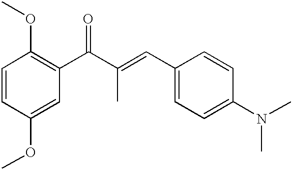 | 23-49% |
| 2 | 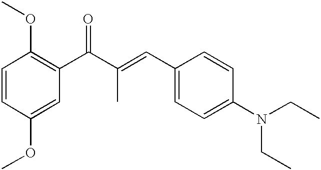 | 20-23% |
| 3 | 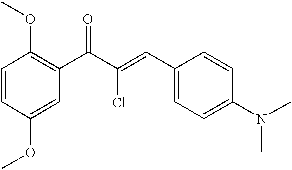 | 25-29% |
| 4 | 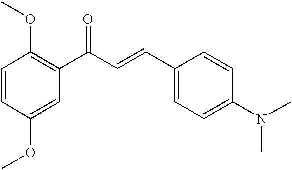 | 18-19% |
| 5 | 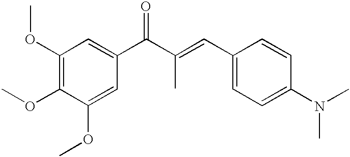 | 15% |
| 6 | 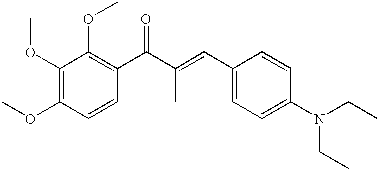 | 35-41% |
| 7 | 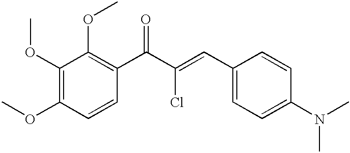 | 22-42% |
| 8 | 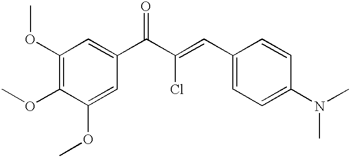 | 23-43% |
| 9 | 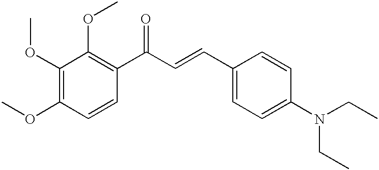 | 19% |
| 10 | 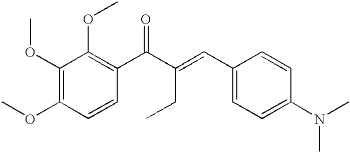 | 15% |
| 11 | 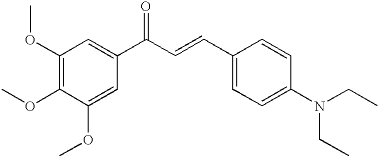 | 20% |
| 12 | 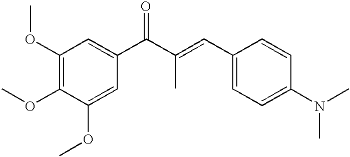 | 29-42% |
| 13 | 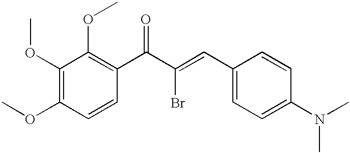 | 23-34% |
| 14 | 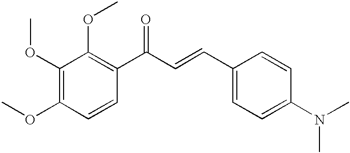 | 18-20% |
| Comparative Example 1 | 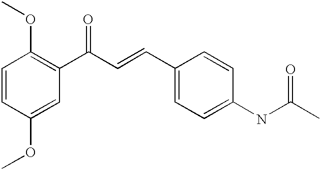 | 2% |
| Comparative Example 2 | 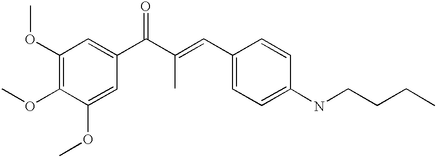 | 6% |

What is claimed is:

1. A process for inhibiting vascularization of a tumor mass comprising administering, in an amount effective to inhibit said vascularization, at least one chalcone of formula (I): 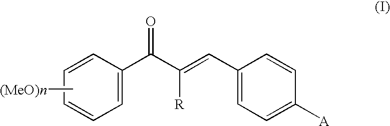
wherein:
R is chosen from a hydrogen atom, a methyl group, an ethyl group, a chlorine atom, and a bromine atom,
A is an NR₁R₂ group, wherein R₁ and R₂, which may be identical or different, are each chosen from a methyl group and an ethyl group, and
n is an integer equal to 2 or 3.

2. A process according to claim 1, wherein said at least one chalcone is chosen from:
1-(2,5-d imethoxyphenyl)-3-[4-(dimethylamino)phenyl]-2-methylprop-2-en-1-one,
1-(2,5-dimethoxyphenyl)-3-[4-(dimethylamino)phenyl]prop-2-en-1-one,
1-(2,5-dimethoxyphenyl)-3-[4-(dimethylamino)phenyl]-2-ethylprop-2-en-1-one,
1-(2,5-dimethoxyphenyl)-3-[4-(dimethylamino)phenyl]-2-bromoprop-2-en-1-one,
1-(2,5-dimethoxyphenyl)-3-[4-(dimethylamino)phenyl]-2-chloroprop-2-en-1-one,
1-(2,5-dimethoxyphenyl)-3-[4-(diethylamino)phenyl]-2-methylprop-2-en-1-one,
1-(2,5-dimethoxyphenyl)-3-[4-(diethylamino)phenyl]prop-2-en-1-one,
1-(2,5-dimethoxyphenyl)-3-[4-(diethylamino)phenyl]-2-ethylprop-2-en-1-one,
1-(2,5-dimethoxyphenyl)-3-[4-(dimethylamino)phenyl]-2-bromoprop-2-en-1-one,
1-(2,5-dimethoxyphenyl)-3-[4-(dimethylamino)phenyl]-2-chloroprop-2-en-1-one,
1-(2,3,4-trimethoxyphenyl)-3-[4-(dimethylamino)phenyl]-2-methylprop-2-en-1-one,
1-(2,3,4-trimethoxyphenyl)-3-[4-(dimethylamino)phenyl]prop-2-en-1-one,
1-(2,3,4-trimethoxyphenyl)-3-[4-(dimethylamino)phenyl]-2-ethylprop-2-en-1-one,
1-(2,3,4-trimethoxyphenyl)-3-[4-(dimethylamino)phenyl]-2-bromoprop-2-en-1-one,
1-(2,3,4-trimethoxyphenyl)-3-[4-(dimethylamino)phenyl]-2-chloroprop-2-en-1-one,
1-(2,3,4-trimethoxyphenyl)-3-[4-(dimethylamino)phenyl]-2-methylprop-2-en-1-one,
1-(2,3,4-trimethoxyphenyl)-3-[4-(diethylamino)phenyl]prop-2-en-1-one,
1-(2,3,4-trimethoxyphenyl)-3-[4-(dimethylamino)phenyl]-2-ethylprop-2-en-1-one
1-(2,3,4-trimethoxyphenyl)-3-[4-(dimethylamino)phenyl]-2-bromoprop-2-en-1-one,
1-(2,3,4-trimethoxyphenyl)-3-[4-(dimethylamino)phenyl]-2-chloroprop-2-en-1-one,
1-(3,4,5-trimethoxyphenyl)-3-[4-(dimethylamino)phenyl]-2-methylprop-2-en-1-one,
1-(3,4,5-trimethoxyphenyl)-3-[4-(dimethylamino)phenyl]prop-2-en-1-one,
1-(3,4,5-trimethoxyphenyl)-3-[4-(dimethylamino)phenyl]-2-ethylprop-2-en-1-one,
1-(3,4,5-trimethoxyphenyl)-3-[4-(dimethylamino)phenyl]-2-bromoprop-2-en-1-one,
1-(3,4,5-trimethoxyphenyl)-3-[4-(dimethylamino)phenyl]-2-chloroprop-2-en -1 -one,
1-(3,4,5-trimethoxyphenyl)-3-[4-(diethylamino)phenyl]-2-methylprop-2-en-1-one,
1-(3,4,5-trimethoxyphenyl)-3-[4-(diethylamino)phenyl]prop-2-en-1-one,
1-(3,4,5-trimethoxyphenyl)-3-[4-(diethylamino)phenyl]-2-ethylprop-2-en-1-one,
1-(3,4,5-trimethoxyphenyl)-3-[4-(dimethylamino)phenyl]bromoprop-2-en-1one, and
1-(3,4,5-trimethoxyphenyl)-3-[4-(dimethylamino)phenyl]-2-chloroprop-2-en-1-one.

3. A pharmaceutical composition comprising, in an amount effective to inhibit vascularization of a tumor mass, at least one chalcone of formula (I): 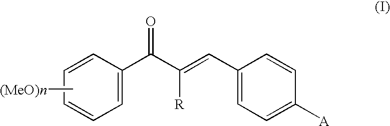
wherein:
R is chosen from a hydrogen atom, a methyl group, an ethyl group, a chlorine atom, and a bromine atom,
A is an NR₁R₂ group, wherein R₁ and R₂, which may be identical or different, are each chosen from a methyl group and an ethyl group, and
n is an integer equal to 2 or 3, and
wherein said composition optionally comprises at least one agent chosen from pharmaceutically acceptable diluents and pharmaceutically acceptable adjuvants.

4. A pharmaceutical composition according to claim 3, wherein said at least one chalcone is chosen from:
1-(2,5-dimethoxyphenyl)-3-[4-(dimethylamino)phenyl]-2-methylprop-2-en-1-one,
1-(2,5-dimethoxyphenyl)-3-[4-(dimethylamino)phenyl]prop-2-en-1-one,
1-(2,5-dimethoxyphenyl)-3-[4-(dimethylamino)phenyl]-2-ethylprop-2-en-1-one,
1-(2,5-d imethoxyphenyl)-3-[4-(dimethylamino)phenyl]-2-bromoprop-2-en-1-one,
1-(2,5-dimethoxyphenyl)-3-[4-(dimethylamino)phenyl]-2-chloroprop-2-en-1-one,
1-(2,5-dimethoxyphenyl)-3-[4-(diethylamino)phenyl]-2-methylprop-2-en-1-one,
1-(2,5-dimethoxyphenyl)-3-[4-(diethylamino)phenyl]prop-2-en-1-one,
1-(2,5-dimethoxyphenyl)-3-[4-(diethylamino)phenyl]-2-ethylprop-2-en-1-one,
1-(2,5-d imethoxyphenyl)-3-[4-(dimethylamino)phenyl]-2-bromoprop-2-en-1-one,
1-(2,5-dimethoxyphenyl)-3-[4-(dimethylamino)phenyl]-2-chloroprop-2-en-1-one,
1-(2,3,4-trimethoxyphenyl)-3-[4-(dimethylamino)phenyl]-2-methylprop-2-en-1-one,
1-(2,3,4-trimethoxyphenyl)-3-[4-(dimethylamino)phenyl]prop-2-en-1-one,
1-(2,3,4-trimethoxyphenyl)-3-[4-(dimethylamino)phenyl]-2-ethylprop-2-en-1-one,
1-(2,3,4-trimethoxyphenyl)-3-[4-(dimethylamino)phenyl]-2-bromoprop-2-en-1-one,
1-(2,3,4-trimethoxyphenyl)-3-[4-(dimethylamino)phenyl]-2-chloroprop-2-en-1-one,
1-(2,3,4-trimethoxyphenyl)-3-[4-(diethylamino)phenyl]-2-methylprop-2-en-1-one,
1-(2,3,4-trimethoxyphenyl)-3-[4-(diethylamino)phenyl]prop-2-en-1-one,
1-(2,3,4-trimethoxyphenyl)-3-[4-(diethylamino)phenyl]-2-ethylprop-2-en-1-one,
1-(2,3,4-trimethoxyphenyl)-3-[4-(dimethylamino)phenyl]-2-bromoprop-2-en-1-one,
1-(2,3,4-trimethoxyphenyl)-3-[4-(dimethylamino)phenyl]-2-chloroprop-2-en-1-one,
1-(3,4,5-trimethoxyphenyl)-3-[4-(dimethylamino)phenyl]-2-methylprop-2-en-1-one,
1-(3,4,5-trimethoxyphenyl)-3-[4-(dimethylamino)phenyl]prop-2-en-1-one,
1-(3,4,5-trimethoxyphenyl)-3-[4-(dimethylamino)phenyl]-2-ethylprop-2-en-1-one,
1-(3,4,5-trimethoxyphenyl)-3-[4-(dimethylamino)phenyl]-2-bromoprop-2-en-1-one,
1-(3,4,5-trimethoxyphenyl)-3-[4-(dimethylamino)phenyl]-2-chloroprop-2-en-1-one,
1-(3,4,5-trimethoxyphenyl)-3-[4-(diethylamino)phenyl]-2-methylprop-2-en-1-one,
1-(3,4,5-trimethoxyphenyl)-3-[4-(diethylamino)phenyl]prop-2-en-1-one,
1-(3,4,5-trimethoxyphenyl)-3-[4-(diethylamino)phenyl]-2-ethylprop-2-en-1-one,
1(3,4,5-trimethoxyphenyl)-3-[4-(dimethylamino)phenyl]-2-bromoprop-2-en-1-one, and
1-(3,4,5-trimethoxyphenyl)-3-[4-(dimethylamino)phenyl]-2-chloroprop-2-en-1-one.
